(19) United States
(12) Patent Application Publication
(10) Pub. No.: US 2005/0131523 A1
(43) Pub. Date: Jun. 16, 2005

(54) Detachable and retrievable stent assembly

(76) Inventors: Mehran Bashiri, San Carlos, CA (US); Mark Phung, Union City, CA (US); Kamal Ramzipoor, Fremont, CA (US); Ajitkumar B. Nair, Fremont, CA (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/809,236

(22) Filed: Mar. 25, 2004

Related U.S. Application Data
(60) Provisional application No. 60/459,696, filed on Apr. 2, 2003.

(51) Int. Cl. A61F 2/06

(52) U.S. Cl. 623/1.15

(57) ABSTRACT

A stent assembly comprises an expandable stent having a first stent backbone which extends from the proximal end of the stent to the distal end of the stent. The stent comprises a first stent back bone oriented in a direction which is substantially parallel to a longitudinal axis of the stent, and a plurality of interconnected first stent members and second stent members. Each of the first stent members is oriented in a substantially longitudinal direction in the unexpanded state and the expanded state. Each of the second stent members is oriented in a substantially longitudinal direction in the unexpanded state and oriented in a substantially circumferential direction in the expanded state. The first stent backbone has a greater column strength than that of the plurality of interconnected stent members.

### CROSS-REFERENCE TO RELATED APPLICATIONS

This Application is a utility Application claiming priority of U.S Provisional Application No. 60/459696, filed Apr. 2, 2003, the entire content of which being incorporated herein by reference.

### STATEMENT REGARDING FEDERALLY SPONSORED RESEARCH

Not Applicable

### BACKGROUND OF THE INVENTION

Stents, grafts, stent-grafts, vena cava filters and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, etc. Stents may be used to reinforce body vessels and to prevent restenosis following angioplasty in the vascular system. They may be self-expanding, mechanically expandable or hybrid expandable.

Stents are generally tubular devices for insertion into body lumens. However, it should be noted that stents may be provided in a wide variety of sizes and shapes. Balloon expandable stents require mounting over a balloon, positioning, and inflation of the balloon to expand the stent radially outward. Self-expanding stents expand into place when unconstrained, without requiring assistance from a balloon. A self-expanding stent may be biased so as to expand upon release from the delivery catheter and/or include a shape-memory component which allows the stent to expand upon exposure to a predetermined condition. Some stents may be characterized as hybrid stents which have some characteristics of both self-expandable and balloon expandable stents.

Due to the branching nature of the human vasculature it is not uncommon for stenoses to form at any of a wide variety of vessel bifurcations. A bifurcation is an area of the vasculature or other portion of the body where a first (or parent) vessel is bifurcated into two or more branch vessels. In some cases it may be necessary to implant multiple stents at the bifurcation in order to address a stenosis located thereon. Alternatively, a stent may be provided with multiple sections or branches that may be deployed within the branching vessels of the bifurcation.

Stents may be constructed from a variety of materials such as stainless steel, Elgiloy, nickel, titanium, nitinol, shape memory polymers, etc. Stents may also be formed in a variety of manners as well. For example a stent may be formed by etching or cutting the stent pattern from a tube or sheet of stent material; a sheet of stent material may be cut or etched according to a desired stent pattern whereupon the sheet may be rolled or otherwise formed into the desired substantially tubular, bifurcated or other shape of the stent; one or more wires or ribbons of stent material may be woven, braided or otherwise formed into a desired shape and pattern. Stents may include components that are welded, bonded or otherwise engaged to one another.

Typically, a stent is implanted in a blood vessel or other body lumen at the site of a stenosis or aneurysm by so-called “minimally invasive techniques” in which the stent is compressed radially inwards and is delivered by a catheter to the site where it is required through the patient's skin or by a “cut down” technique in which the blood vessel concerned is exposed by minor surgical means. When the stent is positioned at the correct location, the stent is caused or allowed to expand to a predetermined diameter in the vessel.

Stents are currently utilized in a variety of applications. However, in some applications, such as for example in procedures involving intracranial placement of a stent, the delivery and placement of the stent is particularly challenging due considerations including access, visualization, control, etc. Many existing stents do not sufficiently address the need for exact placement and/or the need to reposition the stent within a lumen, as may be necessary in some intracranial procedures. Despite the wide variety of stents presently available, there remains a desire to provide stents and stent designs which provide a stent that is capable of precise placement, including the ability to reposition the stent following its expansion.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In light of the above the present invention is directed to a variety of embodiments. In at least one embodiment the invention comprises a stent that is repositionable within a body lumen even after the stent has been expanded.

In at least one embodiment the stent is self-expandable.

In at least one embodiment the stent is inflation expandable.

In at least one embodiment the stent is hybrid expandable.

In at least one embodiment the stent is constructed from a nickel titanium alloy such as nitinol.

In at least one embodiment the stent is at least partially constructed from a cut tube.

In at least one embodiment the stent is at least partially constructed from one or more wires.

In at least one embodiment the stent is configured to deliver one or more therapeutic agents.

In at least one embodiment the stent comprises one or more coatings.

In at least one embodiment the stent defines a back bone, the back bone is a longitudinal component of the stent having one or more physical characteristics different than the remainder of the stent. In some embodiments the back bone comprises a wall thickness that is greater than that of the wall thickness of the remainder of the stent. In some embodiments the variable thickness of the stent may be provided by masking selected portions of the stent prior to microblasting and/or electropolishing the stent. In some embodiments the back bone provides the stent with improved manipulation or ‘push’ characteristics by providing a portion of the stent with a longitudinal component that has a greater column strength than the remainder of the stent. In some embodiments the stent is provided with a plurality of back bone elements. In some embodiments the back bone has one or more components that are oriented in a direction other than in the longitudinal direction of the stent.

In at least one embodiment the stent is at least partially radiopaque. In some embodiments one or more radiopaque markers are engaged to the back bone or are positioned adjacent thereto.

In at least one embodiment the stent has a variable stiffness.

In at least one embodiment, prior to delivery of the stent, the stent is engaged to a push/pull wire, hereinafter referred to as a “push wire”, the push wire being engaged to the stent at a proximal severable junction. In some embodiments the severable junction is non-conductive. In some embodiments the stent is released from the push wire when the severable junction is severed or otherwise disrupted. In some embodiments the severable junction is severed by electrolytic corrosion, mechanical actuation, application of hydraulic pressure, one or more thermal processes, application of electromagnetic energy, etc. The push wire is constructed and arranged to allow a user to manipulate the stent within a lumen even after the stent has been deployed and/or expanded. Once the stent is positioned in a manner and location that is desired the severable junction is severed and the push wire may be withdrawn.

In at least one embodiment the stent is characterized as comprising a plurality of struts that are moveable in a variety of directions when the stent expands. The moveable struts provide the stent with the capability of being folded, collapsed and/or expanded in a manner that aids in minimizing the level of strain on the individual struts. In some embodiments struts are moveable in a direction substantially perpendicular to the longitudinal axis of the stent. In some embodiments struts are moveable in such a manner so as to provide the stent with a predetermined degree of longitudinal foreshortening. In some embodiments the stent does not substantially longitudinally foreshorten when being expanded from an unexpanded state to an expanded state.

In at least one embodiment the stent comprises struts or other elements that are moveable in a longitudinal direction to a greater extent than elements in line with the push wire.

In at least on embodiment the stent is delivered into a body lumen through a catheter. In some embodiments, the stent is configured such that in the folded or collapsed state individual stent components resist tuliping thereby allowing the stent to pass through the catheter with reduced frictional interference.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a longitudinal side view of an embodiment of the invention in a predeployed configuration.

FIG. 2 is a longitudinal side view of the embodiment shown in FIG. 1 depicted during initial deployment.

FIG. 3 is a longitudinal side view of the embodiment shown in FIG. 1 in an initially deployed configuration.

FIG. 4 is a longitudinal side view of the embodiment shown in FIG. 1 in a fully deployed configuration.

FIG. 5 is a longitudinal side view of an embodiment of the invention depicting the assembly in a predeployed configuration.

FIG. 6 is a longitudinal side view of the assembly shown in FIG. 5 wherein the stent is shown being initially deployed from the catheter.

FIG. 7 is a longitudinal side view of the assembly shown in FIG. 6 wherein the stent is shown partially expanded and partially unexpanded during initial deployment from the catheter.

FIG. 8 is a longitudinal side view of the assembly shown in FIG. 7 wherein the stent is shown partially expanded and partially unexpanded during initial deployment from the catheter.

FIG. 9 is a longitudinal side view of the assembly shown in FIG. 8 wherein the stent is shown partially expanded and partially unexpanded during initial deployment from the catheter.

FIG. 10 is a longitudinal side view of the assembly shown in FIGS. 5-9 wherein the assembly is shown in an initially deployed configuration wherein the stent is in a fully expanded state but still engaged to the push wire.

FIG. 11 is a longitudinal side view of the assembly shown in FIGS. 5-10 wherein the assembly is shown in a fully deployed configuration wherein the stent is in a fully expanded state but has been disengaged from the push wire.

FIG. 12 is a longitudinal top down view of an embodiment of the invention, wherein the stent is shown in an expanded state.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

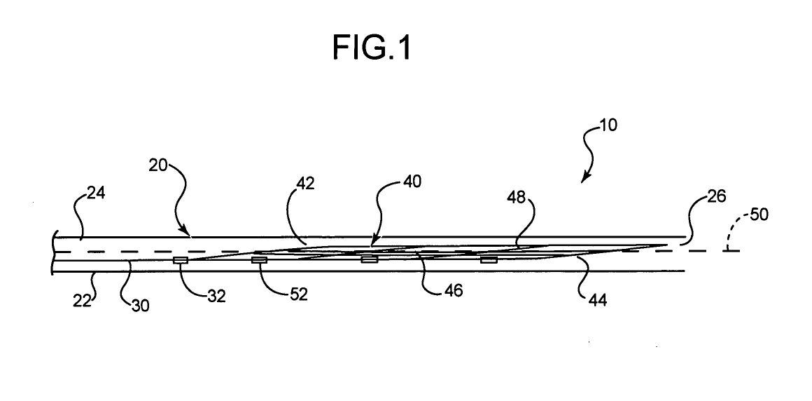

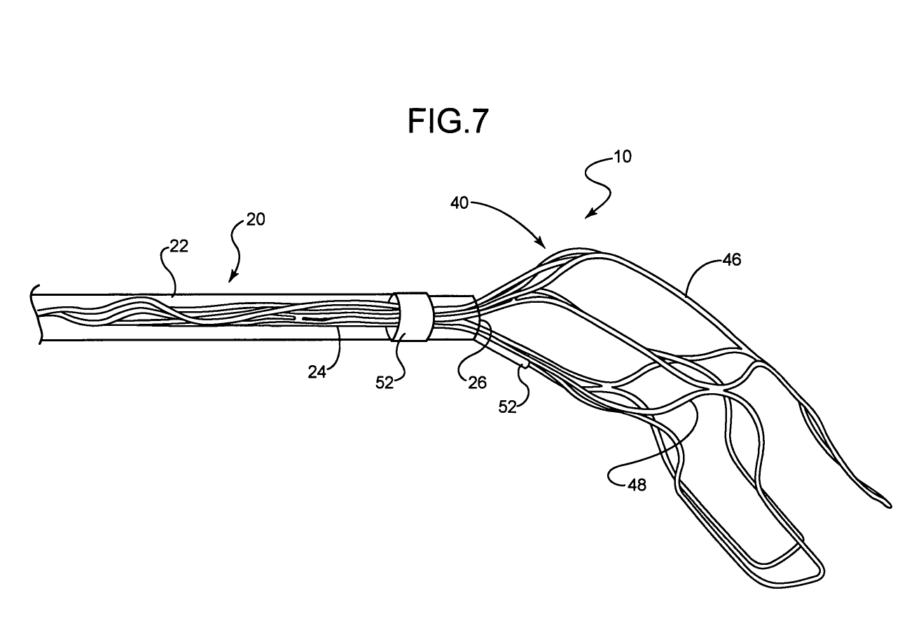

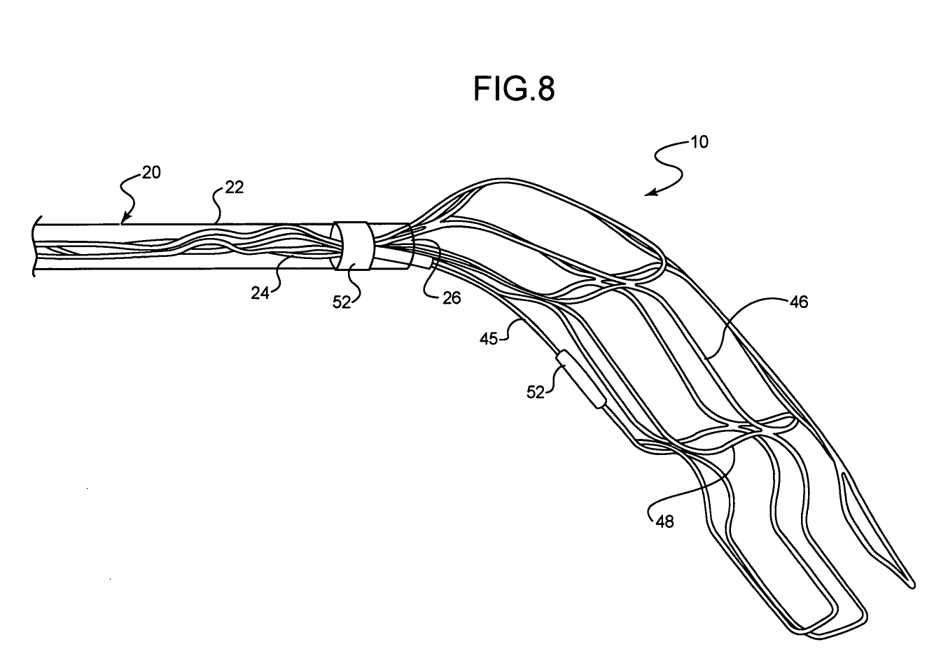

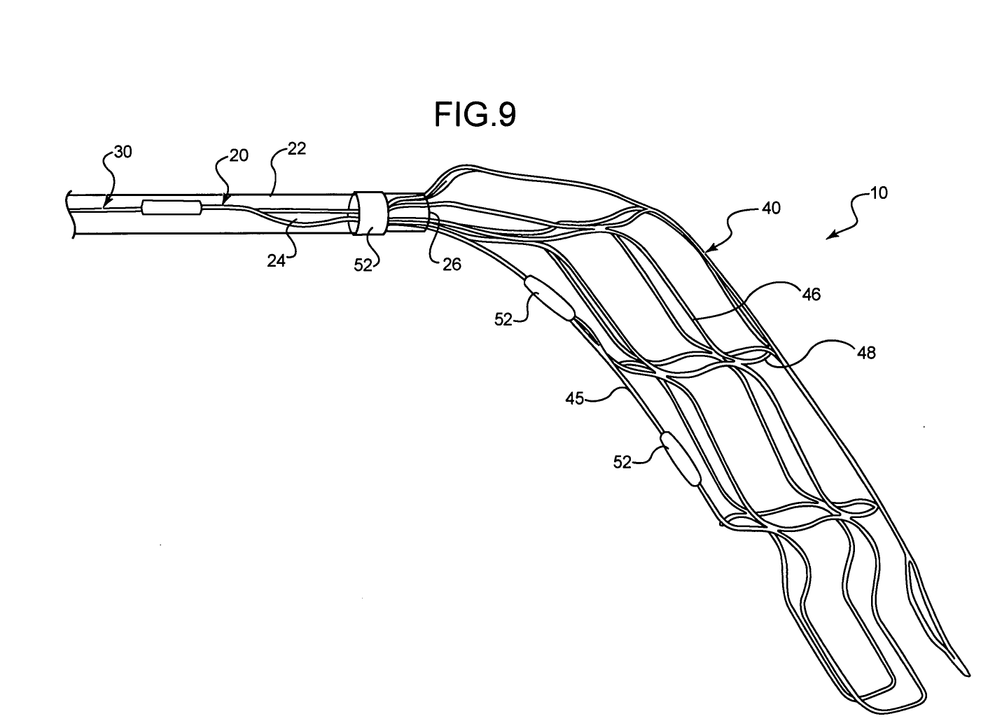

As indicated above the present invention is embodied in a variety of forms. Some examples of some embodiments are depicted in FIGS. 1-11.

In an embodiment of the invention depicted in FIGS. 1-11 an assembly 10 is depicted having a variety of components. The assembly 10 includes a catheter 20 a push wire 30 and an expandable stent 40.

Catheter 20 may be any type of catheter suitable for use in delivering a medical device to a body lumen. In at least one embodiment the catheter is a microcatheter such as may be suitable for use in delivering medical devices to intracranial spaces or vessels. An example of a suitable catheter 20 is the Excelsio™ microcatheter (1018) available from Boston Scientific Corporation of Natick, Mass. Catheters such as those depicted in U.S. Pat. No. 4,739,768, U.S. Pat. No. 4,979,959, U.S. Pat. No. 4,973,493, and U.S. Pat. No. 5,002,582 are also suitable for use in the present invention. It is noted however that other catheter types and designs in addition to those described above may also be suitable for use as catheter 20.

Catheter 20 comprises a catheter shaft 22, which defines a lumen 24. The shaft 22 further defines an opening 26, which is in communication with the lumen 24 at the distal end 28 of the catheter 20.

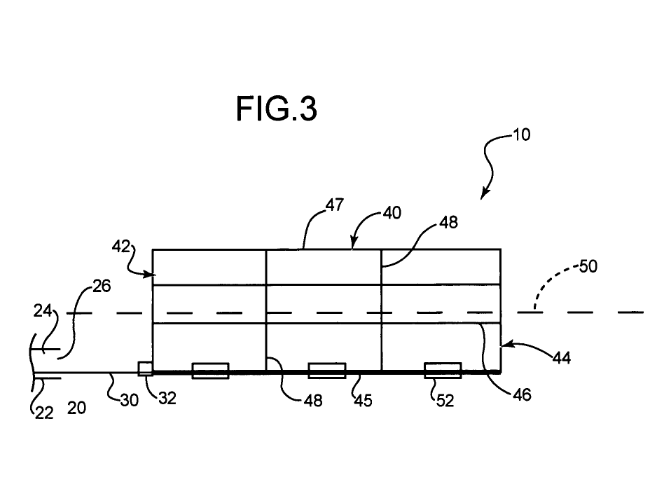

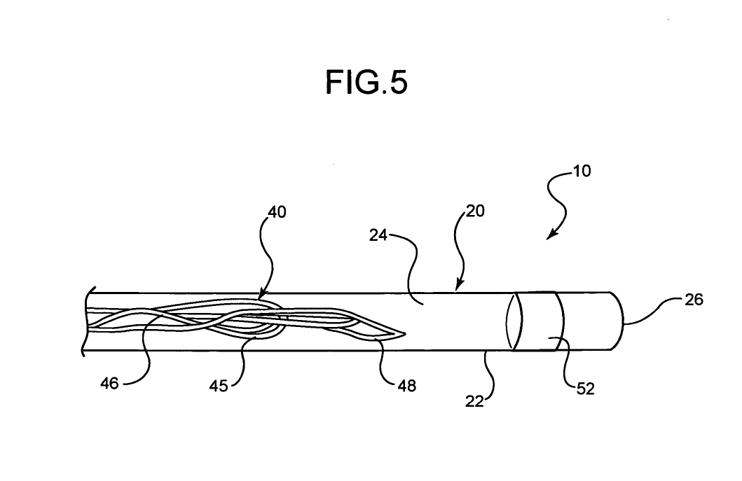

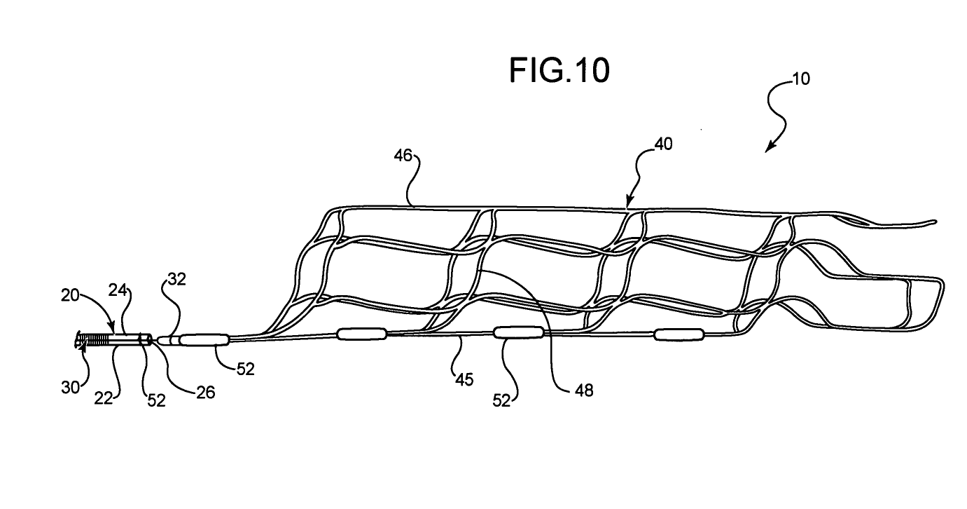

Stent 40 comprises a proximal end region 42 and a distal end region 44. The push wire 30 (not shown in FIG. 5-8) is removably engaged to the proximal end region 42 of the stent. As a result of the engagement of the push wire 30 to the stent 40, prior to fully deploying the stent 40 into a body lumen, the stent 40 may be readily advanced through the catheter lumen 24 by pushing the push wire 30 therethrough. Even after the stent 40 is fully expanded, such as is shown in FIGS. 3 and 10, the stent 40 may be repositioned within the lumen by advancing or withdrawing the push wire 30 to move the stent in a desired direction. In order to better facilitate advancement of the stent 40 through the catheter lumen 24 by pushing the push wire 30, the stent is provided with a unique backbone 45 which is in longitudinal alignment with the push wire 30 and provides greater column strength than the remaining individual components of the stent 40. When engaged to the stent 40 the push wire 30 effectively extends from the backbone 45 or vice versa.

In the embodiment shown in FIGS. 1-11 the stent 40 comprises a backbone 45 and a plurality of first and second stent members or struts 46 and 48. When stent 40 is in the unexpanded state, such as is shown in FIG. 1 and 5, each of the first stent members 46 and second stent members 48 lay down along the backbone 45 such that members 46 and 48 are oriented in a substantially longitudinal direction. As is shown however in FIGS. 3-4 and 10-11 when the stent 40 is in the expanded state, the first members 46 remain oriented in a substantially longitudinal direction relative to the longitudinal axis 50 of the stent 40 while each of the second members 48 attain a substantially circumferential orientation.

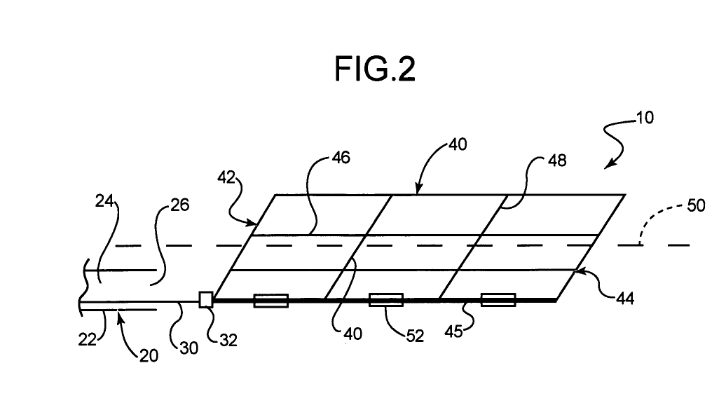

The stent 40 may be self-expandable, inflation expandable, and/or hybrid expandable. Where the stent 40 is inflation expandable, or alternatively in some embodiments where the stent is hybrid expandable, the stent 40, in the unexpanded state, may be disposed about a balloon or other inflation mechanism which expands the stent after the stent is advanced out of the catheter 20.

Where the stent 40 is a self-expandable stent the stent 40 is held in the unexpanded state by the catheter 20 in a predeployed configuration. When the stent 40 is advanced beyond the opening 26 of the catheter 20, or the stent 40 is held in place and the catheter 20 is withdrawn from about the stent 40, the stent 40 will begin to expand such as is shown in FIGS. 2 and 6-9. As is shown by comparing the various sates of expansion shown in FIGS. 1-11, each of the first stent members 46 and each of the second stent members 48 expand relative to the backbone 45. As a result of this type of expansion, the stent 40 has a minimal degree of longitudinal foreshortening. In some embodiments the stent does not longitudinally foreshorten when expanded from the unexpanded state to the expanded state. In some embodiments the degree of longitudinal foreshortening between the unexpanded state and the expanded state is less than half of the circumference of the catheter opening 26. In some embodiments the degree of longitudinal foreshortening between the unexpanded state and the expanded state is less than 5% of the length of the stent.

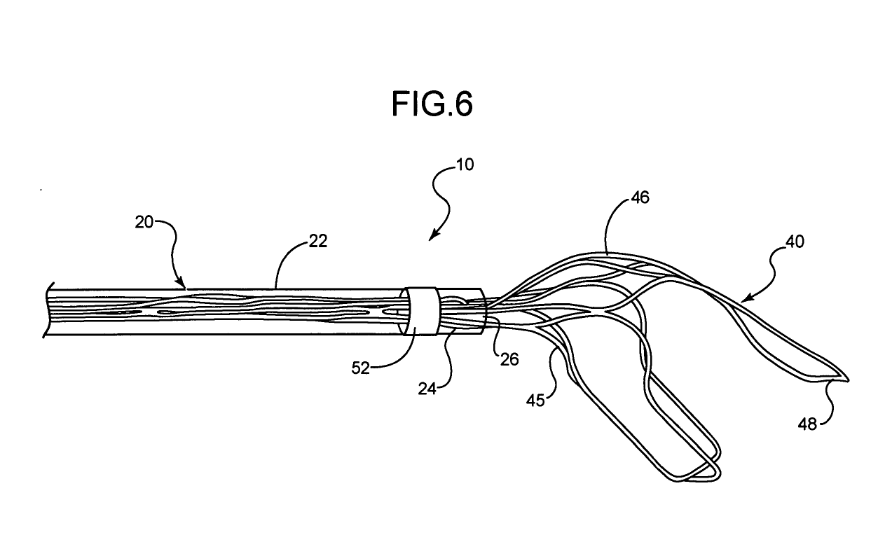

As is shown in FIG. 2, as well as in FIGS. 6-9, as the stent expands the second members 48 move from the more longitudinal orientation shown in FIGS. 1 and 5 to a more circumferential orientation or direction, such as is shown in FIGS. 2-3 and 6-10. The second members 48 may be characterized as ‘standing up’ on the backbone 45. As a result of the second members 48 standing up on the backbone 45, the first members 46 positioned between adjacent second members 48 will be drawn radial away from the backbone 45 while remaining in a substantially longitudinal orientation relative thereto. In at least one embodiment, one or more of the first members remain substantially parallel to the backbone 45 in the expanded state and the unexpanded state.

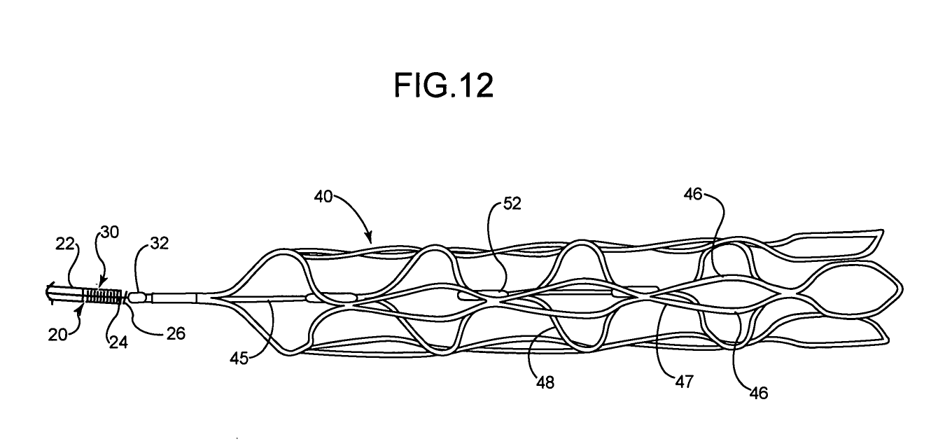

As indicated above the backbone 45 is longitudinally aligned with the push wire 30, and when the push wire 30 is engaged to the stent 40, the push wire extends from the backbone 45 or vice versa. In some embodiments the stent comprises a second backbone 47. The second backbone 47 may have an equal or different column strength to that of the first backbone 45. Backbones 45 and 47 may each be comprised of a single longitudinally oriented strut having a greater thickness than the thickness of the first or second members 46 and 48. Alternatively, one or more of the backbones 45 and 47 may be comprised of one or more first stent members 46 such as is best shown in FIG. 12. In some embodiments, in the unexpanded state the first backbone 45 is circumferentially and longitudinally offset from the second backbone 47, but in the expanded state the first backbone 45 is not longitudinally offset from the second backbone. In some embodiments, in the unexpanded state the first backbone 45 is circumferentially and longitudinally offset from the second backbone 47 to a predetermined degree but in the expanded state degree to which the first backbone 45 and the second backbone 47 are longitudinally offset is reduced.

Backbones 45 and 47 are typically substantially straight in configuration, tacking into account the need of the stent 40 to curve and bend within tortuous vessels or other body lumens. In some embodiments however, one or more of the backbones 45 and 47 may be configured to have one or more curved portions. However, regardless of the number and type of curved and/or straight portions within the first backbone 45, the first backbone 45 remains oriented in a substantially longitudinal direction in order to maintain pushability of the stent. In some embodiments the backbone 45 is parallel to the longitudinal axis 50 of the stent 40.

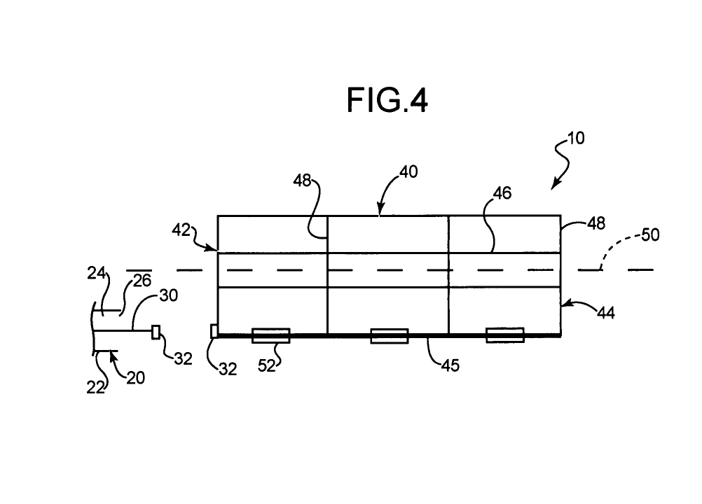

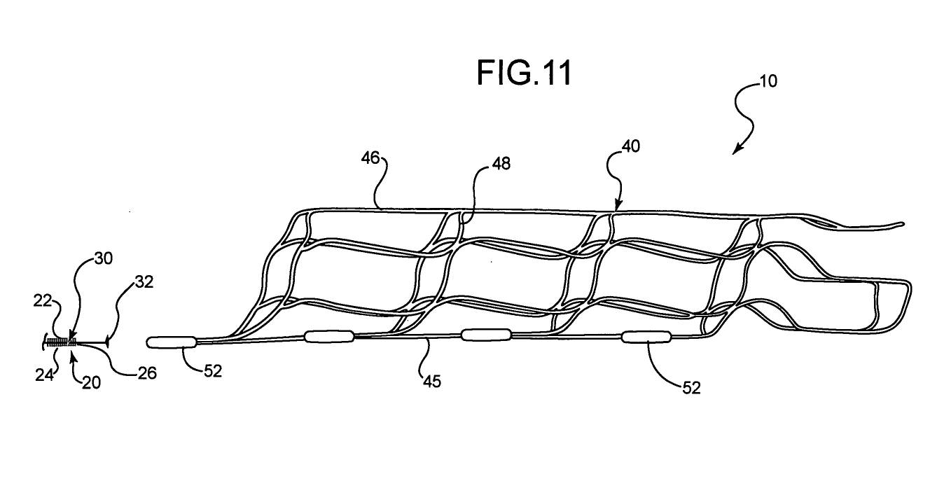

As indicated above, the push wire 30 is removably engaged to the stent 40. In some embodiments the push wire 30 is engaged to the stent at a severable junction 32. After the stent 40 has been advanced through the catheter 20, and the stent is expanded, such as is shown in FIGS. 3 and 6-9, the severable junction 32 is severed, such as is shown in FIG. 4 and FIG. 11 and the stent 40 is released from the push wire 30. In a fully deployed configuration such as is shown in FIGS. 4 and 11, the stent 40 is fully expanded and maintains its position in the lumen by frictional engagement with the lumen wall, the catheter 20 and guide wire 30 may be withdrawn from the lumen following full deployment of the stent 40.

Prior to severance of the severable junction 32 but following release of the stent 40 from the catheter 20 the assembly 10 is in the initially deployed configuration such as is shown in FIG. 10. In this configuration the stent 40 may be moved in a longitudinally in a proximal or distal direction as a result of the continued engagement to the guide wire 30. Even when the stent 40 is in the expanded state the stent may be manipulated in this manner. Such continued engagement with the push wire 30 allows the stent 40 to be retracted back into the catheter lumen 24 if it is desired to significantly reposition the stent 40 or abort the deployment entirely.

Junction 32 may be severed using any of a variety of different methods including, but not limited to, bioabsorption, electrolytic corrosion, mechanical actuation, hydraulic pressure, thermal processes, electromagnetic energy, and so forth as described above. Other methods of detachment known to those of skill in the art but not described herein may also be employed in releasing the device of the present invention.

Some examples of severable junctions which may be employed in the present invention are described, for example, in U.S. Pat. No. 5,122,136; U.S. Pat. No. 5,354,295; U.S. Pat. No. 5,540,680; U.S. Pat. No. 5,855,578; U.S. Pat. No. 5,895,385; U.S. Pat. No. 5,925,037; U.S. Pat. No. 5,944,714; U.S. Pat. No. 5,947,963; U.S. Pat. No. 5,976,126; U.S. Pat. No. 6,010,498; U.S. Pat. No. 6,066,133; U.S. Pat. No. 6,083,220; U.S. patent application Ser. No. 10/230803, filed Aug. 29, 2002, and entitled Device for Closure of a Vascular Defect and Method for Treating Same; U.S. patent application Ser. No. 10/231391, filed Aug. 29, 2002 and entitled Device and Method for Treatment of a Vascular Defect, each of which is incorporated by reference herein in its entirety.

In some embodiments of the invention one or more components of the assembly 10, including the catheter 20, push wire 30 and/or stent 40 may be at least partially radiopaque. In some embodiments, the backbone 45 of the stent 40 has one or more radiopaque markers 52 engaged thereto. Markers 52 may comprise a coating of radiopaque material, a radiopaque band or fastener engaged to the backbone 45 or any other radiopaque mechanism suitable for use in a stent delivery system. In some embodiments such as is best shown in FIG. 5 a portion of the catheter 20 may be equipped with a radiopaque marker 52 adjacent to the opening 26, however one or more markers may be positioned anywhere desired on the catheter 20 or other assembly component.

As is known in the art stents may have a variety of configuration, methods of manufacture, materials, etc. In the present invention the stent 40 may be at least partially constructed from a suitable stent material including but not limited to: stainless steel, Elgiloy, nickel, titanium, and alloys thereof. Other materials include shape memory polymers and shape memory metals such as nitinol.

In some embodiments the stent 40 may be constructed by cutting the desired stent pattern of a stent 40 having one or more backbones 45 and 47 and a plurality of first stent members 46 and second stent members 48 as described above, from a tube of suitable stent material.

In some embodiments the stent 40 may be constructed from one or more wires of suitable stent material, wherein at least one wire is arranged to form a stent 40 having one or more backbones 45 and 47 and a plurality of first stent members 46 and second stent members 48 as described above.

In some embodiments the stent 40 is constructed in accordance with any desired or known construction technique. The backbone 45 may then be formed by masking the area of the stent which corresponds to the position of the backbone. The unmasked portion of the stent is then microblasted, electropolished, and/or otherwise processed to reduce the thickness of the unmasked portion of the stent while maintaining the thickness of the masked portion. Following such processing the masking is removed and the stent 40 is provided with a backbone 45 with a greater columnar strength than the remaining portions of the stent.

The various embodiments of the stents described herein may include one or more coatings and/or other delivery mechanisms which comprise one or more therapeutic agents, cellular materials, polymeric agents, drugs, etc.

The therapeutic agent may be non-genetic or genetic. Suitable non-genetic therapeutic agents include anti-thrombogenic agents such as heparin, heparin derivatives, urokinase, and PPack (dextrophenylalanine proline arginine chloromethylketone), anti-proliferative agents such as enoxaprin, angiopeptin, or monoclonal antibodies capable of blocking smooth muscle cell proliferation, hirudin, and acetylsalicylic acid, anti-inflammatory agents such as dexamethasone, prednisolone, corticosterone, budesonide, estrogen, sulfasalazine, and mesalamine, antineoplastic/antiproliferative/anti-miotic agents such as paclitaxel, 5-fluorouracil, cisplatin, vinblastine, vincristine, epothilones, endostatin, angiostatin and thymidine kinase inhibitors, anesthetic agents such as lidocaine, bupivacaine, and ropivacaine, anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides, vascular cell growth promoters such as growth factor inhibitors, growth factor receptor antagonists, transcriptional activators, and translational promoters, vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol-lowering agents; vasodilating agents; and agents which interfere with endogenous vascoactive mechanisms.

Suitable genetic materials include anti-sense DNA and RNA, DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin like growth factor, cell cycle inhibitors including CD inhibitors, thymidine kinase (“TK”) and other agents useful for interfering with cell proliferation, the family of bone morphogenic proteins (“BMP's”), BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Any of BMP-2, BMP-3, BMP-4, BMP-5, BMP-6 and BMP-7 are particularly desirable. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Suitable cellular materials include cells of human origin (autologous or allogeneic) or from an animal source (xenogeneic), genetically engineered if desired to deliver proteins of interest at the transplant site. The delivery media can be formulated as needed to maintain cell function and viability.

Suitable polymer coating materials include polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof, coatings from polymer dispersions such as polyurethane dispersions (BAYHDROL®, etc.), fibrin, collagen and derivatives thereof, polysaccharides such as celluloses, starches, dextrans, alginates and derivatives, hyaluronic acid, squalene emulsions. Desirably, polyacrylic acid, available as HYDROPLUS® (Boston Scientific Corporation, Natick, Mass.), and described in U.S. Pat. No. 5,091,205, the disclosure of which is hereby incorporated herein by reference, may be used. Also desirably, the polymer may be a copolymer of polylactic acid and polycaprolactone. Other materials include selected medical-grade biodegradable materials such as PGA-TMC, Tyrosine-Derived Polycarbonates and arylates, polycaprolactone co butyl acrylate and other co polymers, Poly-L-lactic acid blends with DL-Lactic Acid, Poly(lactic acid-co-glycolic acid), polycaprolactone co PLA, polycaprolactone co butyl acrylate and other copolymers, Tyrosine-Derived Polycarbonates and arylate, poly amino acid, polyphosphazenes, polyiminocarbonates, polydimethyltrimethylcarbonates, biodegradable CA/PO₄'s, cyanoacrylate, 50/50 DLPLG, polydioxanone, polypropylene fumarate, or polydepsipeptides.

Other suitable coatings include macromolecules such as chitosan and Hydroxylpropylmethylcellulose. Surface erodible materials may also be used. Coatings may also comprise maleic anhydride copolymers, zinc-calcium phosphate and amorphous polyanhydrides.

In some embodiments the stent or one or more portions thereof may be provided with a hydrophilic and/or a hydrophobic coating.

The inventive medical devices may also be provided with a sugar or more generally a carbohydrate and/or a gelatin to maintain the inventive medical devices on a balloon during delivery of the medical device to a desired bodily location. Other suitable compounds for treating the inventive medical devices include biodegradable polymers and polymers which are dissolvable in bodily fluids. Portions of the interior and/or exterior of the inventive medical devices may be coated or impregnated with the compound. Mechanical retention devices may also be used to maintain the inventive medical devices on the balloon during delivery.

The inventive medical devices may also be provided in whole or in part with one or more of the above therapeutic agents, polymeric coatings or the like. Where multiple therapeutic agents are provided, different coatings and/or mechanisms may release the drugs at different rates. For example, one therapeutic agent may be released at a fast rate and another therapeutic agent may be released at a slow rate. Where multiple polymeric coatings are provided, the coatings may degrade or erode at different rates.

In order to facilitate the retention and delivery of one or more therapeutic agents any of the stent embodiments described herein may be provided with a plurality of cavities, micro holes, slits, and/or other surface features such as are known in the art. Such surface features increase or otherwise alter the surface area of the stent to provide the stent with a more optimum agent delivery mechanism. Where the stent is provided with one or more cavities, the cavities may extend partially or entirely through the width of a given stent component. Any of the components of the stent may be provided with one or more cavities.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A stent assembly comprising a stent, the stent having a proximal end and a distal end and being configurable between an unexpanded state and an expanded state, the stent comprising:
a first stent backbone which extends from the proximal end of the stent to the distal end of the stent, the first stent back bone being oriented in a direction which is substantially parallel to a longitudinal axis of the stent; and
a plurality of interconnected first stent members and second stent members, each of the first stent members being oriented in a substantially longitudinal direction in the unexpanded state and the expanded state, each of the second stent members being oriented in a substantially longitudinal direction in the unexpanded state and being oriented in a substantially circumferential direction in the expanded state,
the first stent backbone having a greater column strength than the plurality of interconnected stent members.

2. The assembly of claim 1 wherein the first stent backbone is comprised of a plurality of first stent members.

3. The assembly of claim 1 wherein the first stent backbone has a predetermined thickness and each of the plurality of interconnected first stent members and second stent members have a predetermined thickness, the predetermined thickness of the first stent backbone being greater than the predetermined thickness of each of the plurality of interconnected first stent members and second stent members.

5. The assembly of claim 1 wherein further comprising a second stent backbone, at least a portion of the second stent backbone being substantially parallel to the longitudinal axis of the stent in the unexpanded state and the expanded state.

6. The assembly of claim 5 wherein the second backbone is comprised of a plurality of first stent members.

7. The assembly of claim 5 wherein the second backbone is comprised of at least two longitudinally adjacent first stent members.

8. The assembly of claim 7 wherein at least a portion of each of the longitudinally adjacent first stent members are spaced apart from one another.

9. The assembly of claim 1 further comprising a push wire, the push wire having a proximal end and a distal end, the distal end of the push wire being removeably engaged to a proximal end of the stent, the first backbone extending from the distal end of the push wire.

10. The assembly of claim 9 wherein the push wire is a thermally conductive.

11. The assembly of claim 9 wherein the push wire is electrically conductive.

12. The assembly of claim 9 wherein the push wire is removeably engaged to the stent at a severable junction, the stent being released from the push wire when the severable junction is severed.

13. The assembly of claim 9 wherein at least a portion of the severable junction is bioabsorbable, the stent being released from the push wire when the at least a portion of the severable junction is bioabsorbed.

14. The assembly of claim 12 wherein the severable junction is constructed and arranged to be severable by at least one mechanism selected from the group consisting of: electrolytic corrosion, mechanical actuation, application of hydraulic pressure, application of thermal energy, application of electromagnetic energy and any combination thereof.

15. The assembly of claim 9 comprising a predeployed configuration, an initially deployed configuration and a fully deployed configuration, in the predeployed configuration the stent in the unexpanded state being in mechanical communication with the push wire, in the initially deployed configuration the stent in the expanded state is in mechanical communication with the push wire, in the deployed configuration the stent in the expanded state is mechanically independent from the push wire.

16. The assembly of claim 15 wherein the assembly is constructed and arranged to be configurable from the predeployed configuration to the initially deployed configuration and from the initially deployed configuration to the fully deployed configuration.

17. The assembly of claim 16 wherein the assembly is constructed and arranged to be configurable from the initially deployed configuration to the predeployed configuration.

18. The assembly of claim 17 further comprising a catheter, the catheter comprising a catheter shaft, the catheter shaft defining a lumen, the shaft further defining an opening at a distal end of the catheter, in the predeployed configuration the stent and push wire being moveably contained within the lumen.

19. The assembly of claim 18 wherein in the initially deployed configuration at least a portion of the push wire is and the stent are free of the lumen.

20. The assembly of claim 19 wherein when the assembly is configured from the predeployed configuration to the initially deployed configuration at least a portion of the push wire and the stent are advanced through the opening at the distal end of the catheter.

21. The assembly of claim 1 wherein the stent is a therapeutic coated stent.

22. The assembly of claim 1 wherein the stent is at least partially constructed of a shape memory material.

23. The assembly of claim 1 wherein the stent is at least partially constructed of nitinol.

24. The assembly of claim 1 wherein the first back bone is at least one wire.

25. The assembly of claim 1 wherein the plurality of interconnected first stent members and second stent members comprise at least one wire.

26. The assembly of claim 3 wherein the stent is at least partially constructed from a tube of stent material.

27. The assembly of claim 1 wherein adjacent interconnected first stent members and second stent members form closed loops.

28. The assembly of claim 1 wherein at least one of the plurality of interconnected first stent members and second stent members comprise at least one substantially curved portion.

29. The assembly of claim 1 wherein at least one of the plurality of interconnected first stent members and second stent members comprise at least one substantially straight portion.

30. The assembly of claim 1 wherein the first back bone comprises at least one substantially curved portion.

31. The assembly of claim 1 wherein the first back bone comprises at least one substantially straight portion.

32. The assembly of claim 5 wherein the second back bone comprises at least one substantially curved portion.

33. The assembly of claim 5 wherein the second back bone comprises at least one substantially straight portion.

34. The assembly of claim 1 wherein the stent is at least partially radiopaque.

35. The assembly of claim 1 wherein at least a portion of the push wire is radiopaque.

36. The assembly of claim 12 wherein the severable junction is at least partially radiopaque.

37. The assembly of claim 1 further comprising at least one radiopaque marker the at least one radiopaque marker being engaged to at least one of the push wire, the first back bone, at least one of the first stent member, and at least one second stent member.

38. The assembly of claim 37 wherein the at least one radiopaque marker comprises a plurality of radiopaque markers.

39. The assembly of claim 5 wherein the first back bone comprises a proximal end and a distal end and the second back bone comprises a proximal end and a distal end, in the unexpanded state the proximal end of the first back bone and the proximal end of the second backbone are longitudinally and circumferentially offset and the distal end of the first back bone and the distal end of the second backbone are longitudinally and circumferentially offset.

40. The assembly of claim 39 wherein in the expanded state the proximal end of the first back bone and the proximal end of the second backbone are longitudinally and circumferentially offset and the distal end of the first back bone and the distal end of the second backbone are longitudinally and circumferentially offset.

41. The assembly of claim 3 wherein the stent is at least partially constructed from a tube of stent material.

42. The assembly of claim 41 wherein the stent is constructed by a method comprising the following steps:
providing a tube of stent material;
cutting a predetermined pattern into the tube of stent material, the predetermined pattern including the first back bone and the plurality of interconnected first stent members and second stent members;
masking an area of the tube of stent material corresponding the position of the first back bone;
removing a predetermined amount of material from at least one area of the stent that is not masked.

43. The assembly of claim 42 wherein the step of removing the predetermined amount of material from at least one area of the stent that is not masked further comprises the step of:
providing the predetermined thickness of the first stent backbone and the predetermined thickness of each of the plurality of interconnected first stent members and second stent members by microblasting the at least one area of the stent that is not masked.

44. The assembly of claim 42 wherein the step of removing the predetermined amount of material from at least one area of the stent that is not masked further comprises the step of:
providing the predetermined thickness of the first stent backbone and the predetermined thickness of each of the plurality of interconnected first stent members and second stent members by electropolishing the at least one area of the stent that is not masked.

45. The assembly of claim 18 wherein at least a portion of the catheter is at least partially radiopaque.

46. The assembly of claim 45 further comprising at least one radiopaque marker, the at least one radiopaque marker being adjacent to the distal end of the catheter.
